(19) United States
(12) Patent Application Publication
(10) Pub. No.: US 2017/0007150 A1
(43) Pub. Date: Jan. 12, 2017

(54) MICROWAVE TOMOGRAPHY APPARATUS AND METHOD THEREOF

(71) Applicant: Electronics and Telecommunications Research Institute, Daejeon (KR)

(72) Inventors: Soon Ik JEON, Daejeon (KR); Bo Ra KIM, Daejeon (KR); Jang Yeol KIM, Daejeon (KR); Hyuk Je KIM, Daejeon (KR); Seong Ho SON, Daejeon (KR); Kwang Jae LEE, Daejeon (KR); Jong Moon LEE, Daejeon (KR); Hyung Do CHOI, Daejeon (KR)

(73) Assignee: Electronics and Telecommunications Research Institute, Daejeon (KR)

(21) Appl. No.: 15/090,066

(22) Filed: Apr. 4, 2016

(30) Foreign Application Priority Data
Jul. 10, 2015 (KR) 10-2015-0098699

(51) Int. Cl. A61B 5/05; A61B 5/00

(52) U.S. Cl. CPC A61B 5/0507; A61B 5/0073; A61B 5/4312; A61B 5/708; A61B 2562/0271; A61B 2576/02

(57) ABSTRACT

According to the exemplary embodiment of the present invention, a microwave tomography apparatus is an apparatus which measures microwave tomograph of a subject which is inserted into a medium container including: a plurality of antennas which is located in the medium container and transmits and receives an electromagnetic wave; a plurality of transceivers which, when a radio wave signal transmitted from one of the plurality of antennas is simultaneously received by the remaining antennas of the plurality of antennas, measures intensity and phase information of the radio wave signal received from the remaining antennas; and a controller which generates an image using the values measured by the plurality of transceivers.

### CROSS-REFERENCE TO RELATED APPLICATIONS

This application claims priority to and the benefit of Korean Patent Application No. 10-2015-0098699 filed in the Korean Intellectual Property Office on Jul. 10, 2015, the entire contents of which are incorporated herein by reference.

### TECHNICAL FIELD

The present invention relates to a microwave tomography apparatus and a method thereof, and more particularly, to a measuring apparatus which transmits a microwave to a portion of a human tissue including a breast to diagnose whether there is abnormality in the tissue.

### BACKGROUND ART

As breast cancer patients are rapidly increased, a necessity of a safe and convenient apparatus for diagnosing the breast cancer is rapidly increased. A breast cancer imaging apparatus (a microwave tomography apparatus) using an electromagnetic wave is developed to diagnose the breast cancer. The breast cancer imaging apparatus using the electromagnetic wave irradiates an electromagnetic wave (microwave) onto a variable imaging subject and then restores an image to diagnose whether a patient has a cancer. That is, when the electromagnetic wave which is output from an aperture of an antenna passes through an imaging subject, a breast cancer imaging apparatus obtains size and phase information of a scattered signal and restores an image through an inverse scattering analysis and diagnoses whether a patient has a cancer from a restored image based on the information.

A configuration and an operation of the breast cancer imaging apparatus of the related art will be described in more detail. The breast cancer imaging apparatus includes a monopole antenna in a medium container, a transmitter which transmits a radio wave signal to the antenna, a plurality of receivers which receives the radio wave signal from the antenna, a switching circuit which selects an antenna to transmit or receive a signal, and a signal processor which processes a received signal.

That is, the breast cancer imaging apparatus of the related art includes the switching circuit between the antenna and the transmitter/receiver so that several signal paths are generated and signal interference is also generated between the transmitted signal and the received signal due to signal leakage. Therefore, measuring quality may be lowered.

The breast cancer imaging apparatus of the related art uses a switching circuit, so that receivers are selected one by one to be sequentially operated for one measurement. Therefore, when several reception antennas are provided, a long measuring time is required.

The breast cancer imaging apparatus of the related art transmits and irradiates the microwave onto a portion of a human tissue including the breast and receives a varying radio wave. Therefore, there is no consideration for elements which secure and maintain a quality of measurement and imaging analysis is simply performed only using radio wave measurement values, so that complexity of image analysis is high and it takes a long time to analyze the image.

### RELATED ART DOCUMENT

### Patent Document

Korean Unexamined Patent Application Publication No. 2010-0072600

### SUMMARY OF THE INVENTION

An exemplary embodiment of the present invention has been made in an effort to provide a technique which prevents signal interruption due to signal leakage between a transmitted signal and a received signal in a microwave tomography apparatus.

An exemplary embodiment of the present invention has also been made in an effort to shorten a measurement time by simultaneously operating a plurality of receivers for one measurement in a microwave tomography apparatus.

An exemplary embodiment of the present invention has also been made in an effort to measure a profile coordinate value of a subject in advance to use the profile coordinate value only to calculate a region in the profile of the subject while substituting a characteristic value of a known medium to a region outside the profile of the subject with respect to the profile coordinate value of the subject during imaging analysis of the entire space, thereby reducing a region to be analyzed to minimize complexity of imaging analysis.

Technical objects of the present invention are not limited to the aforementioned technical objects and other technical objects which are not mentioned will be apparently appreciated by those skilled in the art from the following description.

An exemplary embodiment of the present invention provides a microwave tomography apparatus which is an apparatus which measures microwave tomograph of a subject which is inserted into a medium container including: a plurality of antennas which is located in the medium container and transmits and receives an electromagnetic wave; a plurality of transceivers which, when a radio wave signal transmitted from one of the plurality of antennas is simultaneously received by the remaining antennas of the plurality of antennas, measures intensity and phase information of the radio wave signal received from the remaining antennas; and a controller which generates an image using the values measured by the plurality of transceivers.

The controller may control to perform primary measurement which measures the intensity and phase information of the radio wave signal in a state when the subject is not present in the medium container and control to perform secondary measurement of the intensity and phase information of the radio wave signal in a state when the measuring subject is inserted into the medium container.

The plurality of transceivers may be connected to the plurality of antennas by one to one correspondence.

The apparatus may further include a surface measuring unit which measures a profile coordinate value of the measuring subject to transmit a measuring result to the controller.

The controller may analyze an image using a first measurement value by the primary measurement, a second measurement value by the secondary measurement, and the profile coordinate value.

The plurality of antennas may include a circularly coupled single antenna.

The microwave tomography apparatus according to an exemplary embodiment of the present invention may further include: a temperature sensor which measures a temperature in the medium container; a temperature heater which consistently maintains the temperature in the medium container; a temperature pump which convects the medium to maintain the temperature in the medium container; and a temperature controller which interworks with the controller to control operations of the temperature sensor, the temperature heater, and the temperature pump.

The microwave tomography apparatus according to an exemplary embodiment of the present invention may further include: a level sensor which measures an amount of medium in the medium container; a medium supply pump which supplies the medium into the medium container; a rotatable driving motor which rotates the plurality of antennas; an up-down driving motor which drives the plurality of antennas up and down; and a driving controller which interworks with the controller to control the level sensor, the medium supply pump, the rotatable driving motor, and the up-down driving motor.

The controller may interwork with the driving controller to control to perform the primary measurement and the secondary measurement while controlling rotation or up-down position of the plurality of antennas.

Another exemplary embodiment of the present invention provides a microwave tomography method which is a method for measuring microwave tomography of a subject through a medium container which includes a plurality of antennas and a plurality of transceivers which corresponds to the plurality of antennas one to one, including: performing primary measurement on a radio wave signal in a state when the subject is not inserted into the medium container; measuring a profile of the subject in a state when the subject is inserted into the medium container; performing secondary measurement on the radio wave signal in a state when the subject is inserted into the medium container; and performing image analysis using the primary measuring result, the profile measuring result, and the secondary measuring result.

The method may further include performing primary initialization which initializes an apparatus before the primary measurement; and performing secondary initialization considering that the subject is inserted into the medium container before the secondary measurement.

The primary initialization and the secondary initialization may set a temperature and a level in the medium container, and a parameter for rotation or up-down position of the antenna.

In the performing of primary measurement or the performing of secondary measurement, when one of the plurality of transceivers transmits a radio wave signal to the plurality of antennas, the remaining transceivers of the plurality of transceivers may simultaneously receive the radio wave signal to measure the radio wave signal.

In the performing of primary measurement or the performing of secondary measurement, the remaining transceivers may measure an intensity and a phase of the radio wave signal.

According to the present invention, a switching circuit is not provided, so that a signal interruption problem due to signal leakage is solved, thereby achieving a measuring quality with a high signal to noise ratio.

According to the present invention, a plurality of transceivers is operated for one measurement, so that a measuring time is shortened. Therefore, an influence by which an electric characteristic of the apparatus varies as time goes is reduced, to improve a measuring quality.

According to the present invention, a measuring condition of a measuring apparatus is consistently maintained by a temperature sensor, a temperature heater, a temperature pump, a level sensor, a medium supply pump, and a rotatable driving motor, thereby improving measuring reliability.

According to the present invention, a profile coordinate value of a human tissue located in a medium container is measured to be used for imaging analysis, so that analysis complexity and analysis time are reduced and the image quality may be improved.

### BRIEF DESCRIPTION OF THE DRAWINGS

FIG. 1 is a diagram of a microwave tomography apparatus according to an exemplary embodiment of the present invention.

FIG. 2 is a flowchart illustrating a microwave tomography method according to an exemplary embodiment of the present invention.

It should be understood that the appended drawings are not necessarily to scale, presenting a somewhat simplified representation of various features illustrative of the basic principles of the invention. The specific design features of the present invention as disclosed herein, including, for example, specific dimensions, orientations, locations, and shapes will be determined in part by the particular intended application and use environment.

In the figures, reference numbers refer to the same or equivalent parts of the present invention throughout the several figures of the drawing.

### DETAILED DESCRIPTION

Hereinafter, some embodiments of the present invention will be described in detail with reference to the accompanying drawings. When reference numerals denote components in the drawings, even though the like parts are illustrated in different drawings, it should be understood that like reference numerals refer to the same parts. In describing the embodiments of the present invention, when it is determined that the detailed description of the known configuration or function related to the present invention may obscure the understanding of embodiments of the present invention, the detailed description thereof will be omitted.

In describing components of the exemplary embodiment of the present invention, terminologies such as first, second, A, B, (a), (b), and the like may be used. However, such terminologies are used only to distinguish a component from another component but nature, a sequence or an order of the component is not limited by the terminologies. If it is not contrarily defined, all terminologies used herein including technological or scientific terms have the same meaning as those generally understood by a person with ordinary skill in the art. Terminologies which are defined in a generally used dictionary should be interpreted to have the same meaning as the meaning in the context of the related art but are not interpreted as ideal or excessively formal meaning if they are not clearly defined in the present invention.

The present invention discloses a microwave tomography technique which transmits and irradiates a microwave onto a part of a measuring subject including a breast (human tissue) and receives a variable electromagnetic wave to measure an intensity and a phase of a signal, analyzes a measuring result to image a radio wave characteristic of the human tissue, and diagnoses whether the tissue is abnormal based on a difference of the radio wave characteristics between tissues. Further, in the present invention, a microwave is used as an example of an electromagnetic wave. However, the electromagnetic wave is not limited to the microwave and includes an electromagnetic wave having various frequency bands.

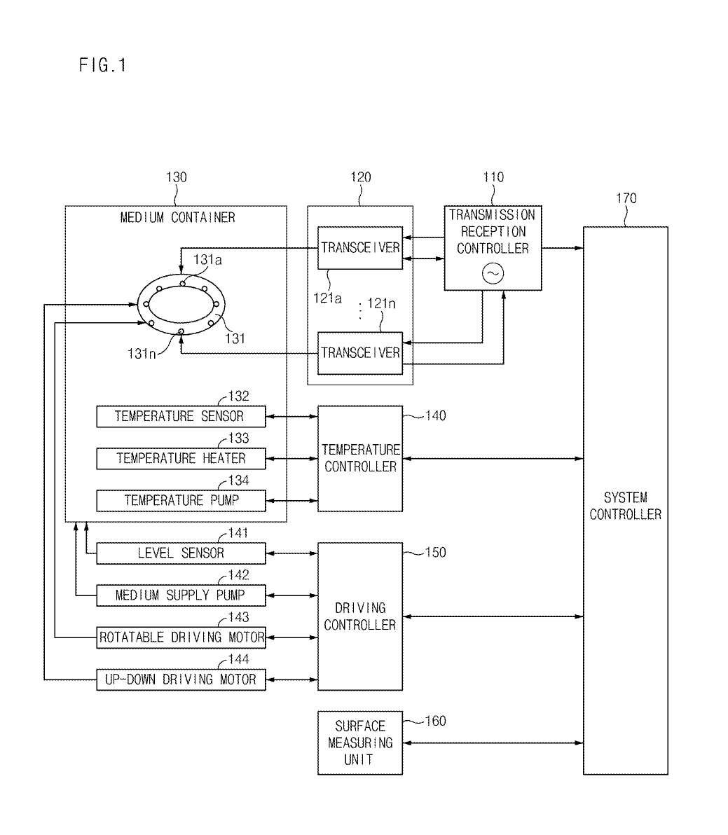

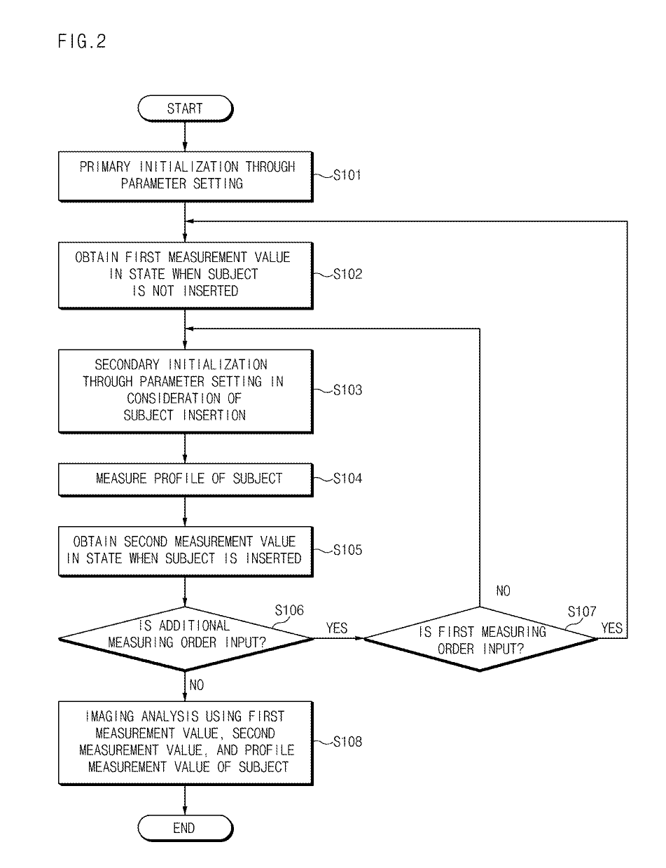

Hereinafter, exemplary embodiments of the present invention will be specifically described with reference to FIGS. 1 to 2.

FIG. 1 is a diagram of a microwave tomography apparatus according to an exemplary embodiment of the present invention.

A microwave tomography apparatus according to an exemplary embodiment of the present invention includes a transmission/reception controller 110, a transceiver array 120, a medium container 130, an antenna 131, a temperature sensor 132, a temperature heater 133, a temperature pump 134, a temperature controller 140, a level sensor 141, a medium supply pump 142, a rotatable driving motor 143, an up-down driving motor 144, a driving controller 150, a surface measuring unit 160, and a system controller 170.

The transmission/reception controller 110 generates a reference frequency signal to the transceiver array 120 and controls transmission/reception of all transceivers 121 and 122.

The transceiver array 120 generates a microwave signal using the reference frequency signal which is received from the transmission/reception controller 110 and transmits the generated microwave signal to the antenna 131. In this case, in the transceiver array 120, N transceivers 121a to 121n may be provided. Further, the N transceivers 121a to 121n in the transceiver array 120 are coupled to N antennas 131a to 131n in the medium container 130 by one to one correspondence. The transceiver array 120 performs frequency conversion processing and signal processing on a signal received from the antenna 131 to measure an intensity and a phase of the signal, thereby generating a first measurement value. For example, when one transceiver among the N transceivers transmits a microwave signal, the signal is output to one of the N antennas and the remaining N−1 transceivers are coupled to the remaining N−1 antennas to simultaneously receive a radio wave signal and measure the intensities and the phases of the signals.

The medium container 130 is filled with a medium so that a human tissue such as a breast which is a measuring subject is inserted therein and the medium container 130 includes the antenna 131 which transmits and receives a radio wave signal, the temperature sensor 132 which maintains a temperature to be a constant temperature as a measuring condition, the temperature heater 133, and the temperature pump 134.

The antennas 131 are located in the medium container 130 and circularly coupled to each other, and N antennas 131a to 131n are arranged on a disc plate with a constant interval. The temperature sensor 132 measures a temperature in the medium container 130 to transmit the measured temperature to the temperature controller 140. The temperature heater 133 supplies heat to maintain a temperature in the medium container 130 in accordance with control of the temperature controller 140. The temperature pump 134 convects the medium to maintain a temperature in the medium container 130 in accordance with control of the temperature controller 140. The temperature controller 140 controls operations of the temperature sensor 132, the temperature heater 133, and the temperature pump 134 in accordance with a temperature in the medium container 130 by the control of the system controller 170.

The level sensor 141 measures an amount of medium of the medium container 130 and the medium supply pump 142 supplies or discharges the medium to or from the medium container 130. The rotatable driving motor 143 is coupled to the antennas 131 which are circularly coupled and rotates a position of the antennas 131. The up-down driving motor 144 is coupled to the antennas 131 which are circularly coupled and moves a position of the antennas 131 up or down. The driving controller 150 controls operations of the level sensor 141, the medium supply pump 142, the rotatable driving motor 143, and the up-down driving motor 144 by the control of the system controller 170.

The surface measuring unit 160 measures a profile coordinate value of a measuring subject which is located in the medium container 130 and transmits the measuring result to the system controller 170. In this case, the profile coordinate value of the measuring subject is a three-dimensional coordinate value and indicates spatial information of the measuring subject.

The system controller 170 is connected to the transmission/reception controller 110, the temperature controller 140, and the driving controller 150 to receive a state signal of the measuring apparatus and outputs a control signal of the measuring apparatus to monitor and control the measuring apparatus of the system. The system controller 170 performs initialization by setting the measuring apparatus to have a predetermined parameter value before measuring the radio wave signal, performs primary measurement through the transceiver array 120 in a state when the measuring subject is not inserted in the medium container 130, and performs secondary measurement through the transceiver array 120 in a state when the measuring subject is inserted in the medium container 130. Further, the system controller 170 performs image analysis using all the primary measurement value, the secondary measurement value, and a surface coordinate value to provide an image. In this case, a general method is used as the image analyzing method. In the present invention, a surface coordinate value of the subject is measured in advance to be directly provided, so that during the imaging analysis of the entire space, with respect to the profile coordinate value of the subject, a known characteristic value of the medium is substituted into a region outside the profile of the subject and only a region in the profile of the subject is calculated. Therefore, a region to be analyzed is reduced, thereby reducing a time to analyze the image and a complexity thereof.

A microwave tomography apparatus with the above-described configuration of the present invention transmits a signal using one of N transceivers and the remaining N−1 transceivers simultaneously receive the signal to measure intensities and phases of the signals, thereby reducing a measuring time. Further, the microwave tomography apparatus does not include a switching circuit, so that signal interruption is prevented. Furthermore, during image analysis, a shape coordinate value of the measuring subject is used, so that complexity of the image analysis is minimized

Hereinafter, a measuring method of a microwave tomography apparatus according to an exemplary embodiment of the present invention will be described in detail with reference to FIG. 2.

A system controller 170 performs primary initialization which sets parameter values of the transmission/reception controller 110, the temperature controller 140, and the driving controller 150 of FIG. 1 to set a parameter value required for each element to initialize the tomography apparatus in step S101. In this case, the parameter includes a level of the medium, a temperature of the medium, an up-down or rotation position of the antenna. When the parameter is set, the temperature sensor 132, the temperature heater 133, the temperature pump 134, the level sensor 141, the medium supply pump 142, the rotatable driving motor 143, and the up-down driving motor 144 are controlled to perform the primary initialization.

Thereafter, while a human tissue is not inserted in the medium container 130, one transceiver 121a among N transceivers of the transceiver array 120 generates a microwave signal from a reference frequency signal and transmits the generated microwave signal to the antenna 131. Next, the remaining N−1 transceivers 121b to 121n simultaneously perform frequency synthesis and signal processing on the signal which is received by the antenna 131 to measure intensities and phases of received radio wave signal. Thereafter, the N−1 transceivers repeatedly measure intensities of the signals while changing a frequency of the received radio wave signal and interwork with the transmission/reception controller 103 and the system controller 170 to repeatedly measure the intensities and phases of the radio wave signals while rotating the antennas 131 through the rotatable driving motor 143. Further, the N−1 transceivers interwork with the transmission/reception controller 103 and the system controller 170 to repeatedly measure the intensities and phases of the radio wave signals while up-down driving the antennas 131 through the up-down driving motor 144 to obtain a first measurement value in step S102.

Thereafter, the system controller 170 resets parameter values of the transmission/reception controller 110, the temperature controller 140, and the driving controller 150 for target measurement to perform secondary initialization for target measurement in step S103. That is, a parameter of the secondary initializing step is similar to a parameter at the time of the primary initializing step of S101, but the parameter is set considering that the medium in the medium container 130 overflows when the measuring subject is inserted into the medium container 130, which is different from the primary initializing step.

Next, in a state when the measuring subject is inserted into the medium container 140, a profile of the measuring subject is measured in step S104. In this case, the profile of the measuring subject is a three-dimensional coordinate value and indicates spatial information of the measuring subject. The profile of the measuring subject is measured in advance because when imaging analysis is performed only using the intensity and the phase information of the radio wave signal through the microwave transmission/reception, it takes a long time and is complex to perform imaging analysis. Therefore, profile information of the measuring subject which is easily obtained is used to perform the imaging analysis, so that a known characteristic value of the medium is substituted to a region outside the profile of the subject with respect to the profile coordinate value of the subject during the imaging analysis of the entire space and only the region in the profile of the subject is calculated. As a result, a region to be analyzed is reduced, thereby minimizing complexity and time.

Thereafter, in a state when the human tissue is inserted into the medium container 130, similarly to the primary measurement of step S102, one transceiver 121a among the N transceivers generates a microwave signal to transmit the microwave signal to the antenna 131 and a radio wave signal which is received by the antenna 131 is simultaneously received by the remaining N−1 transceivers 121b to 121n to perform frequency synthesis and signal processing and measure an intensity and a phase of the signal to obtain a secondary measurement value in step S105. In this case, at the time of the secondary measurement, similarly to the primary measurement, the N−1 transceivers 121b to 121n interwork with the transmission/reception controller 110 and the system controller 170 to repeatedly measure the intensities and phases of the radio wave signal while up-down driving the antennas 131 through the up-down driving motor 144.

Next, the system controller 170 determines whether a user inputs an additional measuring order. When the additional measuring order is not input, the system controller 170 performs imaging analysis using the first measurement value, the second measurement value, and the profile measurement value of the subject in step S108. In this case, when the measuring subject is the breast, if the user wants to continuously measure a right breast after measuring a left breast, the additional measuring order may be input from the user.

In the meantime, when the additional measuring order is input, it is determined whether a first measurement value obtaining request in a state when no measuring subject is inserted in step S102 is input in step S107. That is, an order indicating whether to use the first measurement value in a state when the measuring subject is not inserted or reobtain the first measurement value is input and when the first measurement value is used, steps S103 to S105 are repeated and when the first measurement value is reobtained, the first measurement value is reobtained by returning to step S102.

The control order is a basic exemplary embodiment and various determining equations are input in FIG. 2 to detour or return to elements in the control during start, processing, and end steps.

It will be appreciated that various exemplary embodiments of the present invention have been described herein for purposes of illustration, and that various modifications, changes, and substitutions may be made by those skilled in the art without departing from the scope and spirit of the present invention.

Accordingly, the exemplary embodiments disclosed herein are intended to not limit but describe the technical spirit of the present invention and the scope of the technical spirit of the present invention is not restricted by the exemplary embodiments. The protection scope of the present invention should be interpreted based on the following appended claims and it should be appreciated that all technical spirits included within a range equivalent thereto are included in the protection scope of the present invention.

What is claimed is:

1. A microwave tomography apparatus in a system which measures microwave tomograph of a subject which is inserted into a medium container, the apparatus comprising:
a plurality of antennas which is located in the medium container and transmits and receives an electromagnetic wave;
a plurality of transceivers which, when a radio wave signal transmitted from one of the plurality of antennas is simultaneously received by the remaining antennas of the plurality of antennas, measures intensity and phase information of the radio wave signal received from the remaining antennas; and
a controller which generates an image using the values measured by the plurality of transceivers.

2. The apparatus of claim 1, wherein the controller controls to perform primary measurement which measures the intensity and phase information of the radio wave signal in a state when the subject is not present in the medium container and controls to perform secondary measurement of the intensity and phase information of the radio wave signal in a state when the measuring subject is inserted into the medium container.

3. The apparatus of claim 1, wherein the plurality of transceivers is connected to the plurality of antennas by one to one correspondence.

4. The apparatus of claim 1, further comprising:
a surface measuring unit which measures a profile coordinate value of the measuring subject to transmit a measuring result to the controller.

5. The apparatus of claim 4, wherein the controller analyzes an image by substituting a known characteristic value of the medium to a region outside of the profile of the subject and calculating only a region in the profile of the subject with respect to the profile coordinate value using a first measurement value by the primary measurement, a second measurement value by the secondary measurement, and the profile coordinate value.

6. The apparatus of claim 1, wherein the plurality of antennas is a circularly coupled single antenna.

7. The apparatus of claim 1, further comprising:
a temperature sensor which measures a temperature in the medium container;
a temperature heater which consistently maintains the temperature in the medium container;
a temperature pump which convects the medium to maintain the temperature in the medium container; and
a temperature controller which interworks with the controller to control operations of the temperature sensor, the temperature heater, and the temperature pump.

8. The apparatus of claim 1, further comprising:
a level sensor which measures an amount of medium in the medium container;
a medium supply pump which supplies the medium into the medium container;
a rotatable driving motor which rotates the plurality of antennas;
an up-down driving motor which drives the plurality of antennas up and down; and
a driving controller which interworks with the controller to control the level sensor, the medium supply pump, the rotatable driving motor, and the up-down driving motor.

9. The apparatus of claim 8, wherein the controller interworks with the driving controller to control to perform the primary measurement and the secondary measurement while controlling rotation or up-down position of the plurality of antennas.

10. A method for measuring microwave tomography of a subject through a medium container which includes a plurality of antennas and a plurality of transceivers which corresponds to the plurality of antennas one to one, the method comprising:
performing primary measurement on a radio wave signal in a state when the subject is not inserted into the medium container;
measuring a profile of the subject in a state when the subject is inserted into the medium container;
performing secondary measurement on the radio wave signal in a state when the subject is inserted into the medium container; and
performing image analysis using the primary measuring result, the profile measuring result, and the secondary measuring result.

11. The method of claim 10, further comprising:
performing primary initialization which initializes an apparatus before the primary measurement;
performing secondary initialization considering that the subject is inserted into the medium container before the secondary measurement.

12. The method of claim 11, wherein the primary initialization and the secondary initialization set a temperature and a level in the medium container, and a parameter for rotation or up-down position of the antenna.

13. The method of claim 10, wherein in the performing of primary measurement or the performing of secondary measurement, when one of the plurality of transceivers transmits a radio wave signal to the plurality of antennas, the remaining transceivers of the plurality of transceivers simultaneously receive the radio wave signal to measure the radio wave signal.

14. The method of claim 13, wherein in the performing of primary measurement or the performing of secondary measurement, the remaining transceivers measure an intensity and a phase of the radio wave signal.
